(19) United States
(12) Patent Application Publication
(10) Pub. No.: US 2005/0208558 A1
(43) Pub. Date: Sep. 22, 2005

(54) Detection kits, such as nucleic acid arrays, for detecting the expression or 10,000 or more Drosophila genes and uses thereof

(75) Inventors: J. Craig Venter, Rockville, MD (US); Mark Adams, North Potomac, MD (US); Peter W. D. Li, Brookeville, MD (US); Eugene W. Meyers, Silver Spring, MD (US)

Correspondence Address: CELERA GENOMICS;ATTN: WAYNE MONTGOMERY, VICE PRES, INTEL PROPERTY, 45 WEST GUDE DRIVE, C2-4#20, ROCKVILLE, MD, 20850, US

(73) Assignee: APPLERA CORPORATION, Norwalk, CT (US)

(21) Appl. No.: 11/097,143

(22) Filed: Apr. 4, 2005

Related U.S. Application Data
(63) Continuation of application No. 09/614,150, filed on Jul. 11, 2000, now abandoned.
(60) Provisional application No. 60/191,637, filed on Mar. 23, 2000.
(60) Provisional application No. 60/184,831, filed on Feb. 24, 2000.
(60) Provisional application No. 60/175,693, filed on Jan. 12, 2000.
(60) Provisional application No. 60/173,383, filed on Dec. 28, 1999.
(60) Provisional application No. 60/164,769, filed on Nov. 12, 1999.
(60) Provisional application No. 60/161,932.
(60) Provisional application No. 60/160,191, filed on Oct. 19, 1999.
(60) Provisional application No. 60/157,832.

(51) Int. Cl. C12Q 1/68; C12M 1/34

(52) U.S. Cl. 435/6; 435/287.2

(57) ABSTRACT

The present invention is based on the sequencing and assembly of the Drosophila melanogaster genome. The present invention provides the primary nucleotide sequence of a large portion of the Drosophila melanogaster genome in a series of genomic and predicted transcript sequences. This information is provided in the form of sequences and annotation information and can be used to generate nucleic acid detection reagents and kits such a nucleic acid arrays.

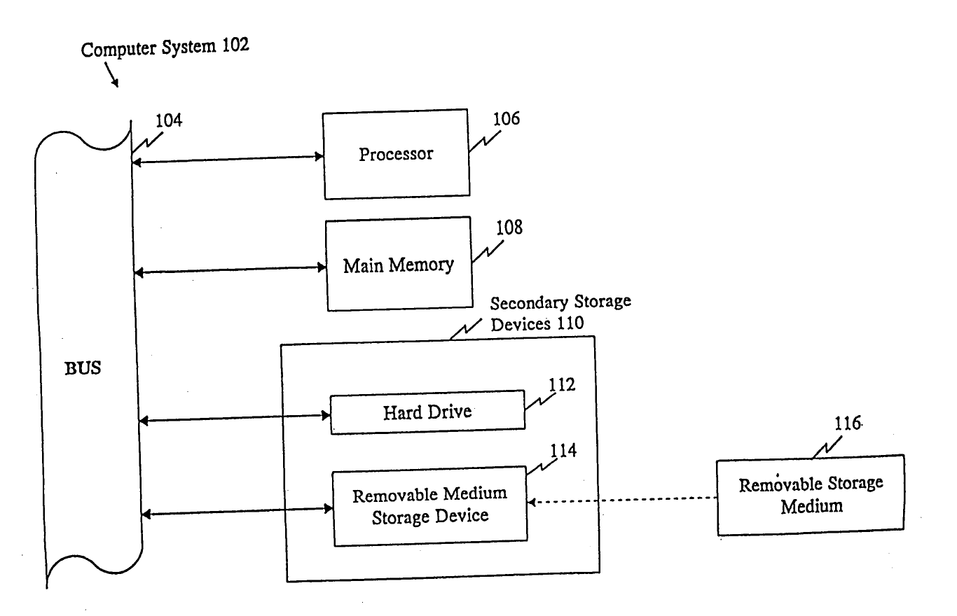

### RELATED APPLICATIONS

The present application claims priority to U.S. Ser. No. 60/157,832, filed Oct. 5, 1999; U.S. Ser. No. 60/160,191, filed Oct. 19, 1999; U.S. Ser. No. 60/161,932, filed Oct. 28, 1999; U.S. Ser. No. 60/164,769, filed Nov. 12, 1999; U.S. Ser. No. 60/173,383, filed Dec. 28, 1999; U.S. Ser. No. 60/175,693, filed Jan. 12, 2000; U.S. Ser. No. 184,831, filed Feb. 24, 2000; U.S. Ser. No. 60/191,637, filed Mar. 23, 2000; and U.S. Ser. No. 09/614,150, filed Jul. 11, 2000.

### FIELD OF THE INVENTION

The present invention is in the field of genomic discovery systems. The present invention specifically provides portions of the Drosophila melanogaster genome in a form that is commercially useful, including detection kits and reagents such as nucleic acid arrays.

### BACKGROUND OF THE INVENTION

Prior to the present invention it was estimated that the Drosophila melanogaster genome was 165 Mb, with about 120 Mb of this being euchromatic. The genome is organized in 4 chromosome pairs and was estimated to contain 10,000-12,000 genes. Model organisms, such as Drosophila melanogaster, share many genes with humans whose sequences and functions have been conserved. In addition to myriad similarities in cellular structure and function, humans and Drosophila share pathways for intercellular signaling, developmental patterning, learning and behavior, as well as tumor formation and metastasis. The present invention advances the art by providing the genomic sequence (SEQ ID NO: 1, 4, 7, 10 . . . 43000, 43003, 43006), transcript sequence (SEQ ID NO: 2, 5, 8, 11 . . . 43001, 43004, 43007) and protein coded sequence (SEQ ID NO: 3, 6, 9, 12 . . . 43002, 43005, 43008) for over 11,000 transcripts/genes that had not previously been identified, as well as the 3,000 genes that were known. A total of 14,338 transcripts are provided herein.

Drosophila studies have provided the widest knowledge base available for any single organism; accordingly, developmental biologists use the fly to identify and characterize the activity of genes with similar functions in higher organisms. Despite its small size, the fly is by no means a small developmental problem. Knowledge of the genes involved in the development of the fly provides, to a reasonable approximation, knowledge of the genes involved in the development of other, more complicated organisms such as the worm, the fish, the mouse, and the human. Developmental biology studies the sequential activation and interaction of genes, in relation to developing morphology. Currently in Drosophila, one can begin with a list of genes active in the egg and follow the morphological changes and gene activation through to adulthood. The genes involved in the development of Drosophila, with few exceptions, are the same as those involved in the development of higher organisms.

A major goal in the development of insecticides, therapeutics, and pharmaceutical drugs is to understand and elucidate the molecular mechanisms that govern cell signaling and cell-cell interactions in higher eukaryotes. The primary sequence of the Drosophila genome in a usable form would therefore be invaluable in developing human therapeutic targets and insecticide targets. Not only will the system serve as a basis for gene discovery and validation, the system will aid in the understanding of complex genetic mechanisms that control cell differentiation, proliferation, and death.

Nucleic Acid Arrays and Detection Kits

Oligonucleotide probes have long been used to detect complementary nucleic acid sequences in a nucleic acid of interest (the “target” nucleic acid) in the form of detection kits/reagents. In some assay formats, the oligonucleotide probe is tethered, i.e., by covalent attachment, to a solid support, and arrays of oligonucleotide probes immobilized on solid supports have been used to detect specific nucleic acid sequences in a target nucleic acid. See, e.g., PCT patent publication Nos. WO 89/10977 and 89/11548. In other formats, the detection reagents are supplied in solution.

The development of arraying technologies such as photolithographic synthesis of a nucleic acid array and high density spotting of cDNA products has provided methods for making very large arrays of oligonucleotide probes in very small areas. See U.S. Pat. No. 5,143,854 and PCT patent publication Nos. WO 90/15070 and 92/10092. Microfabricated arrays of large numbers of oligonucleotide probes, called “DNA chips” offer great promise for a wide variety of applications.

The present invention provides nucleic acid arrays and detection kits that are based on the novel sequences of the Drosophila melanogaster genome provided herein.

### SUMMARY OF THE INVENTION

The present invention is based on the sequencing and assembly of the Drosophila melanogaster genome. The present invention provides the primary nucleotide sequence of a large portion of the Drosophila melanogaster genome in a series of genomic (SEQ ID NO: 1, 4, 7, 10 . . . 43000, 43003, 43006) and predicted transcript sequences (SEQ ID NO: 2, 5, 8, 11 . . . 43001, 43004, 43007: See the Sequence Listing and the Figure Sheets for both the genomic and transcript sequences). This information is provided in the form of sequences and annotation information and can be used to generate nucleic acid detection reagents and kits such a nucleic acid arrays.

The present invention provides these nucleotide sequences of the Drosophila melanogaster genome, or a representative fragment thereof, in a form that can be used, analyzed, and commercialized. For example, the present invention provides the nucleic acid sequences as contiguous strings of primary sequences in a form readable by computers, such as recorded on computer readable media e.g., magnetic storage media, such as floppy discs, hard disc storage medium, and magnetic tape; optical storage media such as CD-ROM; electrical storage media such as RAM and ROM; and hybrids of these categories such as magnetic/optical storage media. The present invention specifically provides a CD-R that comprises this sequence information (in the form of a Sequence Listing). Such compositions are useful in the discovery of drug and insecticide targets.

The present invention further provides systems, particularly computer-based systems that contain the primary sequence information of the present invention stored in data storage means. Such systems are designed to identify commercially important fragments of the Drosophila melanogaster genome.

Another embodiment of the present invention is directed to isolated fragments, and collections of fragments, of the Drosophila melanogaster genome. The fragments of the Drosophila melanogaster genome include, but are not limited to, fragments that encode peptides, hereinafter open reading frames (ORFs) and fragments that modulate the expression of an operably linked ORF, hereinafter expression modulating fragments (EMFs). The ORFs are provided in the Sequence Listing and in the File Drosophila_Genes_Transcripts_Proteins, provided on the accompanying CD labeled CL000728CDA, while the EMFs can be identified as being 5′ to these regions (1 KB of genomic sequence found 5′ of each transcript is provided: discussed in detail below).

The present invention further includes kits, such as nucleic acid arrays, detection reagents and microfluidic devices, that comprise one or more fragments of the Drosophila melanogaster genome of the present invention, particularly ORFs. The kits, such as arrays, can be used to track the expression of many genes, even all genes, or rationally selected subsets thereof, contained in the Drosophila melanogaster genome.

The identification of the entire coding set of sequences from the genome of Drosophila melanogaster will be of great value to all laboratories working with this organism and for a variety of commercial purposes. Many fragments of the Drosophila melanogaster genome will be immediately identified by similarity searches against protein and nucleic acid databases and by identifying structural motifs present in protein domains and will be of immediate value to Drosophila melanogaster researchers and for commercial value for the production of proteins or to control gene expression. A specific example concerns secreted proteins, ion channels and G-protein coupled receptors. The biological significance of secreted proteins for controlling cell signaling, differentiation and proliferation is well known. Many of the known human therapeutic proteins have Drosophila melanogaster orthologs. The Drosophila melanogaster genome will serve as a rich source of such therapeutic proteins.

Further, the development of insecticide targets and therapeutic protein therapeutics and protein targets for human intervention typically involves identifying a protein that can serve as a target for the development of a small molecule modulator. Many classes of proteins are well characterized as suitable pharmaceutical drugs (protein therapeutics or modified forms thereof), drug targets and/or insecticide targets. These include, but are not limited to, secreted proteins, GPCRs and ion channels.

### BRIEF DESCRIPTION OF THE FILES CONTAINED ON CD LABELED CL000728CDA

File Drosophila_Genes_Transcripts_Proteins.doc provides the results of detailed computer analysis of the Drosophila Genome. The file provides for every identified gene/coding region:
1) a genomic fragment spanning the gene, including 1 KB of sequence 5′ of the identified ATG start site and 1 KB of sequence 3′ to the termination site, the predicted exon boundaries and predicted start (ATG) site (SEQ ID NO: 1, 4, 7, 10 . . . 43000, 43003, 43006);
2) the predicted transcript sequence of the gene and starting ATG site (SEQ ID NO: 2, 5, 8, 11 . . . 43001, 43004, 43007);
3) the predicted protein sequence (SEQ ID NO: 3, 6, 9, 12 . . . 43002, 43005, 43008);
4) Whether the sequence of the protein or transcript is known in the art (DNA:+ is known DNA:− is unknown.

The size of this file is 118,675 KB and is stored as a MSWord document.

File SEQLIST.TXT provides a copy of the Sequence Listing of the present invention in text (ASCII) format. The file size is 180,628 KB.

### BRIEF DESCRIPTION OF THE FIGURE

The Figure provides a block diagram of a computer system 102 that can be used to implement the computer-based systems of present invention.

### DETAILED DESCRIPTION OF THE PREFERRED EMBODIMENTS

The present invention is based on the sequencing and assembly of the Drosophila melanogaster genome. In this process, the primary nucleotide sequence of over three million nucleic acid fragments, from about 400 to about 600 nucleotides in length, was determined. These fragments were assembled using the Celera Assembler. After assembly, the sequences were analyzed with various computer packages and compared with all external data sources. The results of this analysis was the identification of 14336 predicted gene/transcripts contained in the Drosophila genome. The present invention provides the genomic nucleic acid sequences (including 1 Kb 5′ and 1 Kb 3′ of the gene start and stop sites, (SEQ ID NO: 1, 4, 7, 10 . . . 43000, 43003, 43006)), predicted transcript sequences (SEQ ID NO: 2, 5, 8, 11 . . . 43001, 43004, 43007), and predicted amino acid sequences of all of these encoded protein (SEQ ID NO: 3, 6, 9, 12 . . . 43002, 43005, 43008), See File Drosophila_Genes_Transcripts_Proteins.doc, provided on the accompanying CD labeled CL000728CDA.

The present invention provides the nucleotide sequences of the present invention, or a representative fragment thereof, in a form that can be readily used, analyzed, and interpreted by a skilled artisan. In one embodiment, the sequences are provided as contiguous strings of primary sequence information corresponding to the nucleotide sequences provided in the Figures and/or File Drosophila_Genes_Transcripts_Proteins.doc.

As used herein, a “representative fragment of the nucleotide sequence provided herein refers to any portion of these sequences that are not presently represented within a publicly available database. Preferred representative fragments of the present invention are Drosophila melanogaster open reading frames and expression modulating fragments (ORFs and EMFs respectively, see below).

The nucleotide sequence information provided herein was obtained by sequencing the Drosophila melanogaster genome using a shotgun sequencing method known in the art. The nucleotide sequences provided herein are highly accurate, although not necessarily a 100% perfect, representation of the nucleotide sequence of the Drosophila melanogaster genome.

Using the information provided in herein together with routine cloning and sequencing methods, one of ordinary skill in the art is able to identify, clone and sequence all “representative fragments” of interest including open reading frames (ORFs) encoding a large variety of Drosophila melanogaster proteins. In very rare instances, this may reveal a nucleotide sequence error present in the nucleotide sequence disclosed herein. Thus, once the present invention is made available (i.e., the information in the Sequence Listing and Figures and File Drosophila_Genes_Transcripts_Proteins.doc in a useable form), resolving a rare sequencing error would be well within the skill of the art. Nucleotide sequence editing software is publicly available.

Even if all of the very rare sequencing errors in the sequences herein disclosed were corrected, the resulting nucleotide sequence would still be at least 90% identical, and more likely 99% identical, and most likely 99.99% identical to the nucleotide sequence provided herein.

Thus, the present invention further provides nucleotide sequences that are at least 90% identical, or greater, to the nucleotide sequences of the present invention in a form which can be readily used, analyzed and interpreted by the skilled artisan. Methods for determining whether a nucleotide sequence is at least 90% identical to the nucleotide sequence of the present invention are routine and readily available to the skilled artisan. For example, the well known BLAST algorithm can be used to generate the percent identity of nucleotide sequences.

The present invention further provides a prediction of all of the genes/exons within the Drosophila genome. This information is provided in File Drosophila_Genes_Transcripts_Proteins.doc, provided on the accompanying CD labeled CL000728CDA. The information in this File can be used to generate detection kits, expression arrays, microfluidic devices, individual gene fragments and the like, and in the identification of commercially important genes and gene products (proteins: (SEQ ID NO: 3, 6, 9, 12 . . . 43002, 43005, 43008).

### Computer Related Embodiments

The nucleotide sequences provided in the present invention, a representative fragment thereof, or nucleotide sequences at least 99% identical to these sequences, may be “provided” in a variety of mediums to facilitate use thereof. As used herein, “provided” refers to a manufacture, other than an isolated nucleic acid molecule, that contains a nucleotide sequence of the present invention, i.e., the nucleotide sequences provided in the present invention, a representative fragment thereof, or nucleotide sequences at least 99% identical to these sequences. Such a manufacture provides the Drosophila melanogaster genome or a subset thereof (e.g., a Drosophila melanogaster open reading frame (ORF)) in a form that allows a skilled artisan to examine the manufacture using means not directly applicable to examining the Drosophila melanogaster genome or a subset thereof as it exists in nature or in purified form.

In one application of this embodiment, a nucleotide sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention. One such medium is provided with the present application, namely, the present application contains computer readable medium (CD-R) that has the sequence contigs provided/recorded thereon in ASCII text format in a Sequence Listing.

As used herein, “recorded” refers to a process for storing information on computer readable medium. A skilled artisan can readily adopt any of the presently known methods for recording information on computer readable medium to generate manufactures comprising the nucleotide sequence information of the present invention.

A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and MicroSoft Word, or represented in the form of an ASCII file, stored in a database application, such as OB2, Sybase, Oracle, or the like. A skilled artisan can readily adapt any number of data processor structuring formats (e.g. text file or database) in order to obtain computer readable medium having recorded thereon the nucleotide sequence information of the present invention.

By providing the nucleotide sequences of the present invention, a representative fragment thereof, or nucleotide sequences at least 99% identical to these sequences, in computer readable form, a skilled artisan can routinely access the sequence information for a variety of purposes. Computer software is publicly available which allows a skilled artisan to access sequence information provided in a computer readable medium. The examples which follow demonstrate how software which implements the BLAST (Altschul et at, J. Mol. Biol. 215:403-410 (1990)) and BLAZE (Brutlag et at, Comp. Chem. 17:203-207 (1993)) search algorithms on a Sybase system was used to identify open reading frames (ORFs) within the Drosophila melanogaster genome which contain homology to ORFs or proteins from other organisms. Such ORFs are protein-encoding fragments within the Drosophila melanogaster genome and are useful in producing commercially important proteins such as proteins used as drug or insecticide targets.

The present invention further provides systems, particularly computer-based systems, which contain the sequence information described herein. Such systems are designed to identify commercially important fragments of the Drosophila melanogaster genome.

As used herein, ‘a computer-based system” refers to the hardware means, software means, and data storage means used to analyze the nucleotide sequence information of the present invention. The minimum hardware means of the computer-based systems of the present invention comprises a central processing unit (CPU), input means, out-put means, and data storage means. A skilled artisan can readily appreciate that any one of the currently available computer-based system are suitable for use in the present invention. Such system can be changed into a system of the present invention by utilizing the sequence information provided on the CD-R, or a subset thereof without any experimentation.

As stated above, the computer-based systems of the present invention comprise a data storage means having stored therein a nucleotide sequence of the present invention and the necessary hardware means and software means for supporting and implementing a search means. As used herein, “data storage means” refers to memory which can store nucleotide sequence information of the present invention, or a memory access means which can access manufactures having recorded thereon the nucleotide sequence information of the present invention.

As used herein, “search means” refers to one or more programs that are implemented on the computer-based system to compare a target sequence or target structural motif with the sequence information stored within the data storage means. Search means are used to identify fragments or regions of the Drosophila melanogaster genome which match a particular target sequence or target motif. A variety of known algorithms are disclosed publicly and a variety of commercially available software for conducting search means are available and can be used in the computer-based systems of the present invention. Examples of such software includes, but is not limited to, MacPattern (EMBL), BLASTN and BLASTX (NCBIA). A skilled artisan can readily recognize that any one of the available algorithms or implementing software packages for conducting homology searches can be adapted for use in the present computer-based systems.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that searches for commercially important fragments of the Drosophila melanogaster genome, such as sequence fragments involved in gene expression and protein processing, may be of shorter length.

As used herein, “a target structural motif,” or “target motif,” refers to any rationally selected sequence or combination of sequences in which the sequence(s) is chosen based on a three-dimensional configuration which is formed upon the folding of the target motif. There are a variety of target motifs known in the art. Protein target motifs include, but are not limited to, enzymatic active sites and signal sequences. Nucleic acid target motifs include, but are not limited to, promoter sequences, hairpin structures and inducible expression elements (protein binding sequences).

A variety of structural formats for the input and output means can be used to input and output the information in the computer-based systems of the present invention. A preferred format for an output means ranks fragments of the Drosophila melanogaster genome possessing varying degrees of homology to the target sequence or target motif. Such presentation provides a skilled artisan with a ranking of sequences which contain various amounts of the target sequence or target motif and identifies the degree of homology contained in the identified fragment.

A variety of comparing means can be used to compare a target sequence or target motif with the data storage means to identify sequence fragments of the Drosophila melanogaster genome. In the present examples, implementing software which implement the BLAST and BLAZE algorithms (Altschul et al., J Mol. Biol. 215:403-410 (1990)) was used to identify open reading frames within the Drosophila melanogaster genome. A skilled artisan can readily recognize that any one of the publicly available homology search programs can be used as the search means for the computer-based systems of the present invention.

One application of this embodiment is provided in the figure. The figure provides a block diagram of a computer system 102 that can be used to implement the present invention. The computer system 102 includes a processor 106 connected to a bus 104. Also connected to the bus 104 are a main memory 108 (preferably implemented as random access memory, RAM) and a variety of secondary storage devices 110, such as a hard drive 112 and a removable medium storage device 114. The removable medium storage device 114 may represent, for example, a floppy disk drive, a CD-ROM drive, a magnetic tape drive, etc—A removable storage medium 116 (such as a floppy disk, a compact disk, a magnetic tape, etc.) containing control logic and/or data recorded therein may be inserted into the removable medium storage device 114. The computer system 102 includes appropriate software for reading the control logic and/or the data from the removable medium storage device 114 once inserted in the removable medium storage device 114.

The nucleotide sequences of the present invention may be stored in a well known manner in the main memory 108, any of the secondary storage devices 110, and/or a removable storage medium 116. Software for accessing and processing the genomic sequence (such as search tools, comparing tools, etc.) reside in main memory 108 during execution.

### Biochemical Embodiments

Nucleic Acid Fragments

Another embodiment of the present invention is directed to isolated fragments of the Drosophila melanogaster genome. The fragments of the Drosophila melanogaster genome of the present invention include, but are not limited to, fragments that encode peptides, hereinafter open reading frames (ORFs) and fragments which modulate the expression of an operably linked ORF. Some of these fragments are identified and described in File Drosophila_Genes_Transcripts_Proteins.doc, provided on the accompanying CD labeled CL000728CDA. The isolated nucleic acid molecules of the present invention include, but are not limited to single stranded and double stranded DNA, and single stranded RNA.

As used herein, an “isolated nucleic acid molecule” or an “isolated fragment of the Drosophila melanogaster genome” refers to a nucleic acid molecule possessing a specific nucleotide sequence which has been subjected to purification means to reduce, from the composition, the number of compounds which are normally associated with the composition. A variety of purification means can be used to generated the isolated fragments of the present invention. These include, but are not limited to methods that separate constituents of a solution based on charge, solubility, or size.

In one embodiment, Drosophila melanogaster DNA can be mechanically sheared to produce fragments of about 2 kb, 10 kb, or 15-20 kb in length. These fragments can then be used to generate a Drosophila melanogaster library by inserting them into plasmid vectors (or lambda vectors) using methods well known in the art. Primers flanking, for example an ORF, can then be generated using nucleotide sequence information provided in the present invention. PCR cloning can then be used to isolate the ORF from the Drosophila DNA library. PCR cloning is well known in the art. Thus, given the availability of the present identified gene coding sequences of the Drosophila genome, it is routine experimentation to isolate any ORF, or other fragment of the assembly of the present invention, particularly using the information provided in File Drosophila_Genes_Transcripts_Proteins.doc, provided on the accompanying CD labeled CL000728CDA. Particularly useful is the generation of nucleic acid fragments comprising one or more exons of a gene, particularly those identified herein. Such fragments can be applied to an array, microfluidic device or other detection kit format and used to detect expression of a gene (see below).

As used herein, an “open reading frame,” ORF, means a series of triplets coding for amino acids without any termination codons and is a sequence translatable into protein. A skilled artisan can readily identify ORFs in the Drosophila melanogaster genome using the gene coding sequences provided herein and/or the computer-based systems of the present invention.

As used herein, an “expression modulating fragment,” EMF, means a series of nucleotide molecules which modulates the expression of an operably linked ORF or EMF.

As used herein, a sequence is said to “modulate the expression of an operably linked sequence” when the expression of the sequence is altered by the presence of the EMF. EMFs include, but are not limited to, promoters, and promoter modulating sequences (inducible elements). One class of EMFs are fragments which induce the expression or an operably linked ORF in response to a specific regulatory factor or physiological event.

EMF sequences can be identified within the Drosophila melanogaster genome by their proximity to the ORFs identified using the computer system of the present invention. An intergenic segment, or a fragment of the intergenic segment, from about 10 to 200, 10 to 500 or 10 to 1 kB nucleotides in length, taken 5′ from any one of the ORFs identified in File Drosophila_Genes_Transcripts_Proteins.doc, provided on the accompanying CD labeled CL000728CDA, will modulate the expression of an operably linked 3′ORF in a fashion similar to that found with the naturally linked ORF sequence. As used herein, an “intergenic segment” refers to the fragments of the Drosophila genome which are between two ORF(G) herein described. Alternatively, EMFs can be identified using known EMFs as a target sequence or target motif in the computer-based systems of the present invention.

The presence and activity of an EMF can be confirmed using an EMF trap vector. An EMF trap vector contains a cloning site 5′ to a marker sequence. A marker sequence encodes an identifiable phenotype, such as antibiotic resistance or a complementing nutrition auxotrophic factor, which can be identified or assayed when the EMF trap vector is placed within an appropriate host under appropriate conditions. As described above, an EMF will modulate the expression of an operably linked marker sequence. A more detailed discussion of various marker sequences is provided below.

A sequence which is suspected as being an EMF is cloned in all three reading frames in one or more restriction sites upstream from the marker sequence in the EMF trap vector. The vector is then transformed into an appropriate host using known procedures and the phenotype of the transformed host in examined under appropriate conditions. As described above, an EMF will modulate the expression of an operably linked marker sequence.

The sequences falling within the scope of the present invention are not limited to the specific sequences herein described, but also include allelic and species variations thereof. Allelic and species variations can be routinely determined by comparing the sequence provided in the present invention, or a representative fragment thereof, with a sequence from another isolate of the same species. Furthermore, to accommodate codon variability, the invention includes nucleic acid molecules coding for the same amino acid sequences as do the specific ORFs disclosed herein. In other words, in the coding region of an ORF, substitution of one codon for another that encodes the same amino acid is expressly contemplated.

Any specific sequence disclosed herein can be readily screened for errors by resequencing a particular fragment, such as an ORF, in both directions (i.e., sequence both strands). Alternatively, error screening can be performed by sequencing correspond polynucleotides of Drosophila melanogaster origin isolated by using part or all of the fragments in question as a probe or primer.

Each of the ORFs of the Drosophila melanogaster genome that can be routinely identified using the computer system of the present invention can be used in numerous ways as polynucleotide reagents. The sequences can be used as diagnostic probes or diagnostic amplification primers to detect the expression of a particular gene or groups of genes. This is particularly useful in the form of nucleic acid arrays where 100 or more, 1000 or more, 5000 or more, or even most to all of the ORFs in a single array.

“Nucleotide sequence” refers to a heteropolymer of deoxyribonucleotides. Generally, DNA segments encoding the polypeptides and proteins provided by this invention are assembled from fragments of the Drosophila melanogaster genome or single nucleotides, short oligonucleotide linkers, or from a series of oligonucleotides, to provide a synthetic nucleic acid molecule.

Nucleic Acid Arrays and Detection Reagents

The present invention further provides detection reagents, such as arrays or microarrays, of nucleic acid molecules that are based on the sequence information provided in the present invention and particularly the transcript information (SEQ ID NO: 2, 5, 8, 11 . . . 43001, 43004, 43007) provided in File Drosophila_Genes_Transcripts_Proteins.doc, provided on the accompanying CD labeled CL000728CDA.

As used herein “Arrays” or “Microarrays” refers to an array of distinct polynucleotides or oligonucleotides synthesized on a substrate, such as paper, nylon or other type of membrane, filter, chip, glass slide, or any other suitable solid, or semi-solid support. In one embodiment, the microarray is prepared and used according to the methods described in U.S. Pat. No. 5,837,832, Chee et al., PCT application W095/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et. al., U.S. Pat. No. 5,807,522.

The microarray or detection kit is preferably composed of a large number of unique, single-stranded nucleic acid sequences, usually either synthetic antisense oligonucleotides or fragments of cDNAs, fixed to a solid support. The oligonucleotides are preferably about 6-60 nucleotides in length, more preferably 15-30 nucleotides in length, and most preferably about 20-25 nucleotides in length. For a certain type of microarray or detection kit, it may be preferable to use oligonucleotides that are only 7-20 nucleotides in length. For others, such as cDNA, longer lengths are possible and preferable. These can be of the order of 1 kb or more.

The microarray or detection kit may contain oligonucleotides that cover the known 5′, or 3′, sequence, sequential oligonucleotides which cover the full length sequence; or unique oligonucleotides selected from particular areas along the length of the sequence. Polynucleotides used in the microarray or detection kit may be oligonucleotides that are specific to a gene or genes of interest.

In order to produce oligonucleotides to a known sequence for a microarray or detection kit, the gene(s) of interest (or an ORF identified from the contigs of the present invention) is typically examined using a computer algorithm which starts at the 5′ or at the 3′ end of the nucleotide sequence. Typical algorithms will then identify oligomers of defined length that are unique to the gene, have a GC content within a range suitable for hybridization, and lack predicted secondary structure that may interfere with hybridization. In certain situations it may be appropriate to use pairs of oligonucleotides on a microarray or detection kit. The “pairs” will be identical, except for one nucleotide that preferably is located in the center of the sequence. The second oligonucleotide in the pair (mismatched by one) serves as a control. The number of oligonucleotide pairs may range from two to one million. The oligomers are synthesized at designated areas on a substrate using a light-directed chemical process. The substrate may be paper, nylon or other type of membrane, filter, chip, glass slide or any other suitable solid support.

In another aspect, an oligonucleotide may be synthesized on the surface of the substrate by using a chemical coupling procedure and an ink jet application apparatus, as described in PCT application W095/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more oligonucleotides, or any other number between two and one million which lends itself to the efficient use of commercially available instrumentation.

In other embodiments, the array or detection reagent/kit can be produced by spotting cDNA or other nucleic acid molecule onto the surface of a substrate (See Brown et. al., U.S. Pat. No. 5,807,522). In such use, PCR primers to one or more exons is used to generate a nucleic acid molecule suitable for deposition onto a substrate.

In order to conduct sample analysis using a microarray or detection kit, the RNA or DNA from a biological sample is made into hybridization probes. The mRNA is isolated, and cDNA is produced and used as a template to make antisense RNA (aRNA). The aRNA is amplified in the presence of fluorescent nucleotides, and labeled probes are incubated with the microarray or detection kit so that the probe sequences hybridize to complementary oligonucleotides of the microarray or detection kit. Incubation conditions are adjusted so that hybridization occurs with precise complementary matches or with various degrees of less complementarity. After removal of nonhybridized probes, a scanner is used to determine the levels and patterns of fluorescence. The scanned images are examined to determine degree of complementarity and the relative abundance of each oligonucleotide sequence on the microarray or detection kit. The biological samples may be obtained from any bodily fluids (such as blood, urine, saliva, phlegm, gastric juices, etc.), cultured cells, biopsies, or other tissue preparations. A detection system may be used to measure the absence, presence, and amount of hybridization for all of the distinct sequences simultaneously. This data may be used for large scale correlation studies on the sequences, expression patterns, mutations, variants, or polymorphisms among samples.

Using such arrays, the present invention provides methods to identify the expression of one or more of the ORFs of the present invention. In detail, such methods comprise incubating a test sample with one or more nucleic acid molecules and assaying for binding of the nucleic acid molecule with components within the test sample. Such assays will typically involve arrays comprising most, if not all of the genes in the Drosophila genome, or rationally selected subsets thereof. The genes/transcript (genomic sequences: (SEQ ID NO: 1, 4, 7, 10 . . . 43000, 43003, 43006); transcript sequences: SEQ ID NO: 2, 5, 8, 11 . . . 43001, 43004, 43007) found in the Drosophila genome of the present invention are provided in File Drosophila_Genes_Transcripts_Proteins.doc provided on the accompanying CD labeled CL000728CDA.

Conditions for incubating a nucleic acid molecule with a test sample vary. Incubation conditions depend on the format employed in the assay, the detection methods employed, and the type and nature of the nucleic acid molecule used in the assay. One skilled in the art will recognize that any one of the commonly available hybridization, amplification or array assay formats can readily be adapted to employ the novel fragments of the Drosophila melanogaster genome disclosed herein. Examples of such assays can be found in Chard, T, An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of Enzyme Immunoassays: Laboratory Techniques in Biochemistry and Molecular Biology, Elsevier Science Publishers, Amsterdam, The Netherlands (1985).

The test samples of the present invention include cells, protein or membrane extracts of cells. The test sample used in the above-described method will vary based on the assay format, nature of the detection method and the tissues, cells or extracts used as the sample to be assayed. Methods for preparing nucleic acid extracts or of cells are well known in the art and can be readily be adapted in order to obtain a sample that is compatible with the system utilized.

In another embodiment of the present invention, kits are provided which contain the necessary reagents to carry out the assays of the present invention.

Specifically, the invention provides a compartmentalized kit to receive, in close confinement, one or more containers which comprises: (a) a first container comprising one of the nucleic acid molecules that can bind to a fragment of the Drosophila melanogaster genome disclosed herein; and (b) one or more other containers comprising one or more of the following: wash reagents, reagents capable of detecting presence of a bound nucleic acid. Preferred kits will include detection reagents/arrays/chips/microfluidic devices that are capable of detecting the expression of 1 or more, 10 or more, 100 or more, or 500 or more, 1000 or more, 10,000 or more, or all of the genes expressed in Drosophila, particularly the genes/exons provided in File Drosophila_Genes_Transcripts_Proteins.doc, provided on the accompanying CD labeled CL000728CDA.

In detail, a compartmentalized kit includes any kit in which reagents are contained in separate containers. Such containers include small glass containers, plastic containers, strips of plastic, glass or paper, or arraying material such as silica. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the nucleic acid probe, containers which contain wash reagents (such as phosphate buffered saline, Tris-buffers, etc.), and containers which contain the reagents used to detect the bound probe. One skilled in the art will readily recognize that the previously unidentified ORFs that can be routinely identified using the sequence information disclosed herein can be readily incorporated into one of the established kit formats which are well known in the art, particularly expression arrays.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claim.

1) An isolated nucleic acid detection reagent that is capable of detecting the presence of 1000 or more genes from Drosophila, wherein said genes are selected from the group consisting of SEQ ID NOS:1, 2, 4, 5, 7, 8, 10, 11 . . . 43006, and 43007.

2) The detection reagent of claim 1, wherein said reagent is a nucleic acid array.

3) The array of claim 2, wherein said array is comprised of short oligonucleotides from about 5 to about 100 nucleotides in length.

4) The array of claim 2, wherein said array is comprised of polynucleotides based on the transcript sequences (SEQ ID NO: 2, 5, 8, 11 . . . 43001, 43004, 43007), wherein said polynucleotides are from about 100 to about 1000 nucleotides in length.

5) An isolated nucleic acid detection reagent that is capable of detecting the presence of 2000 or more genes from Drosophila, wherein said genes are selected from the group consisting of SEQ ID NOS:1, 2, 4, 5, 7, 8, 10, 11 . . . 43006, and 43007.

6) The detection reagent of claim 5, wherein said reagent is a nucleic acid array.

7) The array of claim 6, wherein said array is comprised of short oligonucleotides from about 5 to about 100 nucleotides in length.

8) The array of claim 6, wherein said array is comprised of polynucleotides based on the transcript sequences (SEQ ID NO: 2, 5, 8, 11 . . . 43001, 43004, 43007), wherein said polynucleotides are from about 100 to about 1000 nucleotides in length.

9) An isolated nucleic acid detection reagent that is capable of detecting the presence of 5000 or more genes from Drosophila, wherein said genes are selected from the group consisting of SEQ ID NOS:1, 2, 4, 5, 7, 8, 10, 11 . . . 43006, and 43007.

10) The detection reagent of claim 9, wherein said reagent is a nucleic acid array.

11) The array of claim 10, wherein said array is comprised of short oligonucleotides from about 5 to about 100 nucleotides in length.

12) The array of claim 10, wherein said array is comprised of polynucleotides based on the transcript sequences (SEQ ID NO: 2, 5, 8, 11 . . . 43001, 43004, 43007), wherein said polynucleotides are from about 100 to about 1000 nucleotides in length.

13) An isolated nucleic acid detection reagent that is capable of detecting the presence of 10,000 or more genes from Drosophila, wherein said genes are selected from the group consisting of SEQ ID NOS:1, 2, 4, 5, 7, 8, 10, 11 . . . 43006, and 43007.

14) The detection reagent of claim 13, wherein said reagent is a nucleic acid array.

15) The array of claim 14, wherein said array is comprised of short oligonucleotides from about 5 to about 100 nucleotides in length.

16) The array of claim 15, wherein said array is comprised of polynucleotides based on the transcript sequences (SEQ ID NO: 2, 5, 8, 11 . . . 43001, 43004, 43007), wherein said polynucleotides are from about 100 to about 1000 nucleotides in length.
